SEQUENCE LISTING (5 sequences)

SEQ ID NO: 1 - DNA, 2625 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccacggtctccacagcactcctgacagccccgccaaaccagagaagaatgggcatgccaaagaccaccccaagattgccaagatctttgagatccagaccatgcccaatggcaaaacccggacctccctcaagaccatgagccgtagaaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatccttcactgctgactctgctgcctgcccgcacagcagcctgcttcccacctcctgcccaggccagccagcctcacccttgcgaaccgtgagcaggaaggcctgggtggatcggcctcctcttcaccccggcagccctgcagtgttcgcttggctccatgctcctcactgcccgcacaccctcactctgccagggcagtgctagtgagctgggcatggtaccagccctggggtgcccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagaccatgtaaataccagactgcaggttggacccagagattcccaagccaaaaccttagctccctccgcacccgatgtgacctctactttccagctagtccgacccacctcaccccgttacagctccccaagtggtttccacatgctctgagaagaggagccctcatcttgaagggcccaggagggtctatggggagaggaactccttgcctagcccaccctgctgccttctgacggccctgcaatgtatcccttctcacagcacatgctgccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctgcctggcctggccctgccctgaggaaggaggggaagctgcagcttgggagagcccctggggctagactctgtaacatcactatccatgcaccaaactaataaaactttgacgagtcaccttccaggacccctgggtaaaaaaaaaaaaaaa

SEQ ID NO: 2 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 3 - DNA, 25 bp, Artificial Sequence
  Synthetic oligonucleotide primer
cacagccatcctcaaagtgctggtc

SEQ ID NO: 4 - DNA, 20 bp, Artificial Sequence
  Synthetic oligonucleotide primer
gctggccaagttgtctaaat

SEQ ID NO: 5 - DNA, 19 bp, Artificial Sequence
  Synthetic oligonucleotide primer
tggagctgtgaactggact

(19) United States
(12) Patent Application Publication
(10) Pub. No.: US 2010/0151459 A1
(43) Pub. Date: Jun. 17, 2010

(54) MARKER FOR DETECTING THE PROPOSED EFFICACY OF TREATMENT

(76) Inventor: Jun Soo Kwon, Jongno-gu (KR)

Correspondence Address: SUGHRUE MION, PLLC, 2100 PENNSYLVANIA AVENUE, N.W., SUITE 800, WASHINGTON, DC, 20037, US

(21) Appl. No.: 12/514,806

(22) PCT Filed: Jan. 26, 2007
(86) PCT No.: PCT/JP2007/051748

§ 371 Date: May 14, 2009

(51) Int. Cl. C12Q 1/68

(52) U.S. Cl. 435/6

(57) ABSTRACT

The invention relates to a method of identifying a marker responsive to aripiprazole treatment comprising the steps of:
(a) preparing a genomic DNA Dopamine D2 Receptor (DRD2) gene or its complementary strand from a specimen of a subject with a psychiatric disorder;
(b) analyzing a sequence of said genomic DNA or its complementary-strand to determine the genotype of the TaqIA polymorphism in the DRD2 gene; and
(c) determining the proposed high therapeutic effects of aripiprazole treatment on a subject with psychiatric disorder by using the polymorphism as a marker.

### TECHNICAL FIELD

The present invention relates to a method of determining a therapeutic effect of aripiprazole use on patients with psychoneurotic diseases using genetic polymorphism as markers, and oligonucleotides used for the method, and a kit for detection.

### BACKGROUND ART

Drug kinetics, genetic polymorphisms of enzymes and proteins involved in drug reactivity have been rapidly elucidated with the development of genomics. In human genomic analysis, single nucleotide polymorphisms (SNPs) attract attention as the most frequent genetic polymorphism marker.

It is known that SNPs is useful to analyze human genome associated with common diseases and drug response (Brookes, A. J., “The essence of SNPs”, Gene, USA, (1999), 234, 177-186; Cargill, M, et al., “Characterization of single-nucleotide polymorphisms in coding regions of human genes”, Nature Genet., USA, (1999), 22, 231-238; Evans, W. E., & Relling, M. V., “Pharmacogenomics: translating functional genomics into rational therapeutics”, Science, USA, (1999), 286, 487-491).

It is also known that haplotype analysis using a plurality of SNPs is useful to analyze disease susceptibility in disease with complicated genetic factors (Schlaak, J. F., et. al., “Cell-type and Donor-specific Transcriptional Responses to Interferon-α” J. Biol. Chem., (2002) 277, 51, 49428-49437).

Nowadays, proposed are studies for elucidating relationship between certain genetic polymorphism of an individual patient and drug susceptibility/drug response leading to establishment of personalized medicine.

Aripiprazole acts on dopamine D2 receptor (DRD2) as partial dopamine-serotonin agonist, and has a different action mechanism from existing antipsychotic medicine (U.S. Pat. No. 5,006,528).

Aripiprazole is considered to act on DRD2 suppressively when DRD2 is excessively stimulated and also to activate DRD2 when stimulus to DRD2 is lowered, thus stabilizing activity of dopaminergic nerve in brain (Reist C., et al.: Current Pharmacogenomics (2005) 3, 305-317).

Aripiprazole is the first atypical antipsychotic introduced to medical practice with partial dopamine-serotonin agonist properties. As a partial agonist, whether aripiprazole displays an agonist effect or attenuates dopaminergic neurotransmission may depend on regional variations in endogenous dopamine tone. Hence, aripiprazole offers a therapeutic advantage to differentially modulate dopaminergic activity in brain regions in a graded fashion. This mechanism of action is intriguing when considered in the context of the dopamine hypothesis of schizophrenia whereby positive symptoms (e.g. hallucinations and delusions) are associated with increased mesolimbic dopaminergic activity while reduced activity in mesocortical dopaminergic pathways underlies negative symptoms (e.g. avolition and anhedonia) and cognitive deficits. Despite its therapeutic promise, antipsychotic response to aripiprazole is highly variable, and some patients do not respond at all to drug therapy.

Treatment-emergent adverse events associated with aripiprazole include insomnia, anxiety, akathisia or worsening of psychosis in some patients. These observations suggest that the underlying mechanism of action of aripiprazole in psychotic disorders is more complex than what would be anticipated solely by simple partial agonist effects at the dopamine D2 receptor.

For example, while aripiprazole attenuates dopaminergic hyperactivity it does not increase locomotor activity in reserpinized (hypodopaminergic) rats, which is not fully consistent with a partial agonist mode of action.

Reist C., et al report that Aripiprazole can induce a diverse range of effects at dopamine D2 receptors depending on the cellular milieu defined by promiscuous interactions with a host of signaling partners and variability in local G protein complement and concentration. This diversity provides an opportunity to illustrate the importance of integrating data on genetic variation in pharmacokinetic pathways and molecular targets for antipsychotics including biogenic amine receptors and their downstream signaling partners. Theragnostics, a new subspecialty of molecular medicine formed by combination of therapeutics with diagnostics, offers the potential to synthesize different types of biomarkers (DNA and protein-based) in the context of antipsychotic treatment outcomes. Because the dopamine receptor genetic variation is extensively reviewed elsewhere, the inventor discusses the pharmacogenomic significance of variability in genes encoding for the 5-HT1A (HTR1A) and 5-HT2A (HTR2A) receptors and CYP2D6- and CYP3A4-mediated aripiprazole metabolism. (Christopher Reist, et al., Current Pharmacogenomics, Vol. 3, No. 4. (December 2005), pp. 305-317.)

It has been reported that there is an association between predisposition of neuroleptic malignant syndrome and DRD2 TaqIA polymorphism because A1 allele is significantly dominated in the schizophrenia patients having the neuroleptic malignant syndrome compared with the schizophrenia patients not having such a complication in the patients with schizophrenia for A1 and A2 alleles of DRD2 TaqIA genotypes. In Table 1 in the above report, A1/A1 is shown to be a minor type compared with A2/A2 and A2/A1 in the patients with schizophrenia (Am J. Psychiatry, (2001) 158, 1714-1716:Suzuki A., et al.)

Previous studies have demonstrated that subjects with one or two A1 alleles of DRD2 TaqIA polymorphism have lower DRD2 density than those with no A1 allele. The present study aimed to examine whether the DRD2 TaqIA genotypes are related to therapeutic response to nemonapride, a selective dopamine antagonist, in schizophrenic patients. The subjects were 25 acutely exacerbated schizophrenic inpatients who had received no medication for at least 1 month before the study. The fixed dose (18 mg/day) of nemonapride was administered to each patient for 3 weeks. The clinical status was prospectively monitored by the Brief Psychiatric Rating Scale (BPRS) before, and 3 weeks after, the treatment. The TaqIA genotypes (A1 and A2 alleles) were determined by the polymerase chain reaction method. Three patients were homozygous for the A1 allele, 11 were heterozygous for the A1 and A2 alleles, and 11 were homozygous for the A2 allele. The patients with one or two A1 alleles (n=14) showed significantly higher percentage improvement in total BPRS and positive symptoms than those with no A1 allele (n=11) after 3-week treatment while the percentage improvement in other subgrouped symptoms (negative, anxiety-depression, excitement and cognitive symptoms) was similar between the two genotype groups. The present results suggest that the DRD2 TaqIA polymorphism is related to early therapeutic response to nemonapride in schizophrenic patients, possibly by modifying the efficiency of DRD2 antagonism of the drug in the central nervous system.

### DISCLOSURE OF INVENTION

It is a major object of the invention to provide a means for determining a therapeutic effect of aripiprazole for the patients with schizophrenia so as to detect and find subjects for administration who have the possibility to obtain the high therapeutic effect.

The present inventors researched the genetic polymorphism associated with the therapeutic effect of aripiprazole having partial dopamine-serotonin agonist properties on the patients with schizophrenia in order to find the marker for personalized medicine for the patients with schizophrenia, consequently have identified that the minor type (A1/A1) of Taq IA genotypes having no association in the healthy adults is significantly associated with the therapeutic effect compared with the major types (A2/A2, A2/A1) and there is no difference in side effects, have suggested the possibility that the genotype becomes the marker for the personalized medicine for the patients with schizophrenia, have found an identification marker which can previously detect and find subjects for administration who have the possibility to obtain the high therapeutic effect, and have completed the present invention.

(1) A method of identifying a marker responsive to aripiprazole treatment comprising the steps of:
(a) preparing a genomic DNA of Dopamine D2 Receptor (DRD2) gene or its complementary strand from a specimen of a subject with a psychiatric disorder;
(b) analyzing a sequence of said genomic DNA or its complementary strand to determine the genotype of the TaqIA polymorphism in the DRD2 gene; and
(c) determining the proposed high therapeutic effect of aripiprazole treatment on a subject with psychiatric disorder by using the polymorphism as a marker.
(2) The method according to (1), wherein the genotype of TaqIA in the DRD2 gene is at least one genotype selected from the group consisting of A1A1, A1A2 and A2A2.
(3) The method according to (1), wherein the genetic polymorphism of TaqIA in the DRD2 gene is reference SNP ID NO: rs1800497.
(4) The method according to any one of (1) to (3), wherein the psychiatric disorder is selected from Schizophrenia, Schizophreniform disorder, or Schizoaffective disorder.
(5) The method according to any one of (1) to (4), wherein the marker is at least one genetic polymorphism selected from the group consisting of (a) to (c);
(a) a polymorphism that is T/T (A1A1 type);
(b) a polymorphism that is T/C (A1A2 type); and
(c) a polymorphism that is C/C (A2A2 type);
wherein said polymorphism is located at position 32806 in AF050737.
(6) The method according to any one of (1) to (4), wherein the genetic polymorphism is detected through at least one technique selected from the group consisting of direct nucleotide sequencing, allele-specific oligonucleotide (ASO)-dot blot analysis, single nucleotide primer extension assay, PCR-single strand conformation polymorphism (SSCP) analysis, PCR-restriction enzyme fragment length polymorphism (RFLP) analysis, Invader assay, quantative real-time PCR assay, and genetic polymorphism assay employing a mass spectrometer (mass array).
(7) The method according to (6), wherein the genetic polymorphism is detected by using single nucleotide primer extension assay.
(8) The method according to (6) or (7), wherein said genetic polymorphism is detected by using a probe or primer which has an oligoncleotide having the sequence of SEQ NO:3, or at least 10 nucleotides thereof wherein the oligonucleotide sequences defining the 3′terminal position at 1 to a few base from the position of the polymorphism located at position 32806 in AF050737.
(9) The method according to (6), wherein the PCR-restriction enzyme fragment length polymorphism (RFLP) analysis is performed by using the restriction enzyme (Taq I) for detecting T/T, T/C, or C/C located at position 32806 in AF050737.
(10) A method of determining a therapeutic effect of aripiprazole on patients with psychoneurotic diseases comprising the steps of:
(a) preparing a genomic DNA Dopamine D2 Receptor (DRD2) gene or its complementary strand from a specimen of a patient with a psychiatric disorder;
(b) analyzing a sequence of said genomic DNA or its complementary-strand to determine the genotype of the TaqIA polymorphism in the DRD2 gene; and
(c) determining the proposed high therapeutic effects of aripiprazole treatment on a subject with psychiatric disorder by using the polymorphism as a marker.

### BRIEF DESCRIPTION OF THE DRAWINGS

FIG. 1: Total PANSS change after abilify treatment.

### BEST MODE FOR CARRYING OUT THE INVENTION

As used herein, abbreviations of amino acids, peptides, nucleotide sequences, nucleic acids, etc. are according to IUPAC-IUB nomenclature [IUPAC-IUB communication on Biological Nomenclature, Eur. J. Biochem., 138: 9 (1984)], “Guideline for preparation of a specification, etc. including nucleotide sequences or amino acid sequences” (edited by Japan Patent Office), and commonly employed symbols used in the field.

As used herein, “genetic polymorphism” or “polymorphism” refers to a group of alleles located at one gene locus or an individual allele belonging to the group. The polymorphism, where only one base is different, is specifically referred to as Single Nucleotide Polymorphism (SNP). The single nucleotide polymorphism is herein referred to as “SNP”. The term “haplotype” refers to the combination of the multiple polymorphisms in a continuous gene region or gene cluster.

Furthermore herein, the genotype indicates a state of the allele on a locus in a certain genetically polymorphic site. For example, the genotype for SNP at position 32806 in AF050737 is represented by C/T heterozygote or T/T and C/C homozygotes. In the present invention, the wild type homozygote A2/A2 which is the C/C homozygote and the heterozygote A2/A1 which is the C/T heterozygote are represented as the major type, and the homozygote A1/A1 which is the T/T homozygote is represented as the minor type. It is predicted that the subject with psychiatric disorder having the minor type A1/A1 which is T/T of TaqIA genotype has the higher possibility that the significant therapeutic effect is observed by the use of aripiprazole than the subject with psychiatric disorder having the major types A2/A2 or A2/A1 which is C/C or C/T. Therefore, the TaqIA genotype can be used as the identification marker for obtaining the high therapeutic effect by the use of aripiprazole in the patients psychiatric disorder having the minor type A1/A1 which is T/T of the TaqIA genotype.

Further, genomic sequence of human genes disclosed herein is based on nucleotide sequences shown in nucleotide sequence database such as GenBank in each accession number. Furthermore, information on SNP position and human genetic polymorphism used in the invention is also shown as Reference SNP ID number (e.g. rs 1800497) (see, reference SNP (refSNP) Cluster Report; http://www.ncbi.nlm.nih.gov/SNP cluster). Information on SNP is disclosed herein by Reference SNP ID number.

Reference SNP ID number of TaqIA DRD2 polymorphism is rs1800497.

The target SNP of the invention is located at position 32806 in genomic sequence AF050737 which includes a DRD2 gene.

Restriction emzyme TaqI is otherwise known as TthHB8I and TfII. EsaBC3 cleaves the same recognition sequence.

GenBank accession number of DRD2 gene is X51362.

As used herein, the term “gene” encompasses double-stranded DNA, as well as single-stranded DNA (sense strand or antisense strand) constituting the double-stranded DNA. Unless otherwise specified, the gene (DNA) employed in the present invention encompasses double-stranded DNA including human genomic DNA, single-stranded DNA including cDNA (sense strand), single-stranded DNA having a sequence complementary to the sense strand, and fragments thereof. The aforementioned gene (DNA) may include regulatory regions, coding regions, exons, and introns. The term “polynucleotide” encompasses RNA and DNA. The term “DNA” encompasses cDNA, genomic DNA, and synthetic DNA. The term “polypeptide” encompasses its fragments, homologues, derivatives, and mutants. The term “mutant” refers to a naturally occurring allelic substitution, a non-naturally occurring allelic substitution, a mutant obtained through alteration (deletion, substitution, addition, or insertion), and a polynucleotide sequence which does substantially not change the function of the polypeptide encoded by the polynucleotide sequence. Alteration of an amino acid sequence, which may naturally occur through, for example, mutation or post-translational modification, can be artificially performed by introducing mutations into the gene.

The present invention has been completed based on the discovery of the fact that the genetic polymorphism, particularly SNP or SNPs including the genotype in the specific position (gene reported to be present in TaqI restriction enzyme region in dopamine D2 receptor [DRD2] gene) of the human gene is strongly correlated with the therapeutic effect by aripiprazole treatment of psychiatric disorder, and the therapeutic effect on the patients having psychiatric disorder scheduled with aripiprazole treatment can be determined in advance by detecting the genetic polymorphism (genotype at the specific position). In other words, the present invention has been completed by finding that the human specific genetic polymorphism, particularly the specific SNPs can be used as the determination marker of the drug therapeutic effect on those subjected to the aripiprazole treatment.

According to the method of the invention, a therapeutic effect of aripiprazole treatment for a subject with psychiatric disorder can be determined by detecting the specific SNP or SNPs of the subject.

The method of the present invention makes it an essential requirement to detect the polymorphism of the human specific gene of a specimen derived from those subjected to the aripiprazole use, i.e., the genetic polymorphism (genotype) present in the TaqI restriction enzyme region on the dopamine D2 receptor (DRD2) gene.

SNPs to be detected (analyzed) by the method of the present invention, i.e., the genetic polymorphism (or the genotype) correlated with the aripiprazole treatment for the subject having the psychiatric disorder is more specifically the genotype A1/A1 (T/T homozygote: genetic polymorphism in which the genotype is located at position 32806 in genomic sequence AF050737 including DRD2 gene. SNPs to be detected has a reference SNP ID number: rs1800497 which is the genetic polymorphism of TaqIA in dopamine D2 receptor (DRD2) gene.

According to the present invention, by detecting the genetic polymorphism (SNPs and haplotype) and the genotype of the human specific gene, it is possible to give information or means useful for the identification of the therapeutic effect of the aripiprazole treatment on the subject having the psychiatric disorder, elucidation of the functions, and predicted diagnosis of the therapeutic effect of the aripiprazole treatment on the subject having the psychiatric disorder. Also according to the present invention, it is possible to provide the information which is a reason to determine a treatment policy for the patients with the psychiatric disorder, and particularly the important information to determine whether the aripiprazole treatment is strongly recommended or not as the treatment policy for the personalized medicine tailored individually for the patients with the psychiatric disorder.

In the present invention, subjects with psychiatric disorder are treated with aripiprazole including commercially available aripiprazole formulation (e.g. Abilify TM tablet), enteric coating formulation, depot formulation, powder formulation, Rapidly Disintegrating Tablet, oral solution, flashmelt oral tablet. These aripiprazole formulation can be used singly or in combination with another antipsychotics.

Preparation of Human Gene Sample with SNPs

According to the present invention, a genomic DNA sample with Dopamine D2 Receptor gene or its complementary strand from a specimen of a patient with a psychiatric disorder is prepared (step a). The genomic DNA sample contains certain genetic polymorphisms (SNPs), specifically SNPs represented by A1A1 of TaqIA polymorphism in DRD2 gene (T/T homozygote: genetic polymorphism in which the genotype located at position 32806 in genomic sequence AF050737 (Reference SNP ID No. rs1800497) including a DRD2 gene is T/T (genotype)). The genomic DNA be prepared by a conventional method, from a subject (e.g., a patient with psychiatric disorder). The sample may also have its complement strand of the DNA with the genetic polymorphism.

Examples of the source of the aforementioned cDNA or genomic DNA include various cells and tissues having the DRD2 gene with SNPs, and cultured cells derived therefrom. Specific examples of the source include body fluids such as blood (e.g., serum or plasma), saliva, lymph, airway mucus, urine, and semen. The aforementioned source material (serving as a sample) is preferably DNA or genomic DNA derived from a human subject before administration of aripiprazole (which includes the case where other drug has already been administered and before administration of aripiprazole). Isolation of RNA from such a source material, isolation and purification of mRNA, preparation of cDNA, cloning thereof, etc. can be carried out through a conventional method.

According to the method of the invention, genomic sequence of specific human gene or its complement strand (e.g. gene or its complement strand with A1A1 of TaqI DRD2 gene) is prepared from aforementioned genetic sample. The preparation of genetic sample can be readily performed by a generally employed gene engineering technique on the basis of specific sequence data of DRD2 gene as disclosed herein [see, for example, Molecular Cloning 2nd Ed, Cold Spring Harbor Lab. Press (1989); or Zoku Seikagaku Jikken Koza “Idenshi Kenkyuho I, II, III” edited by The Japanese Biochemical Society (1986)].

Stated more specifically, genomic DNA or mRNA is extracted, by a conventional method, from a subject (e.g., a patient with psychiatric disorder who has SNPs of TaqI DRD2 gene) using a restriction enzyme, probe which may include a specific polymorphism site of DRD2 TaqIA polymorphism [see, for example, Proc. Natl. Acad. Sci., U.S.A., 78, 6613 (1981); or Science, 222, 778 (1983)], to thereby prepare a target genomic sequence of DRD2 gene. Specifically, the target genomic sequence of DRD2 gene is prepared by preparing a probe including the SNP site which can be selectively hybridized with DNA sequence of target SNP, followed by conducting a single nucleotide primer extension assay, Invader assay, quantative real-time PCR assay, etc.

The primers employed for screening may be a forward primer or reverse primer designed on the basis of the neighboring nucleotide sequence data of the DRD2 TaqIA polymorphism. Such primers can be synthesized by a conventional method, for example, an automated synthesis apparatus. The probe for screening is generally a labeled probe. However, the screening probe may be an unlabeled probe, so long as it can specifically bind to a directly or indirectly labeled reagent. The labeling reagent and labeling technique of such a probe or ligand have already been well known in the field. Examples of the labeling reagent include radioactive labeling reagents, biotin, fluorescent dyes, chemiluminescent reagents, enzymes (e.g., luciferase), and antibodies, which can be incorporated through a known labeling technique such as nick translation, random priming, or kinase treatment.

The thus-extracted DNA or mRNA including the SNP site of DRD2 TaqIA polymorphism can be amplified by a gene amplification method. This gene amplification enables easier and accurate detection through the method of the present invention. Examples of the gene amplification method include PCR (Saiki, R. K., Bugawan, T. L., et al., Nature, 324, 163-166 (1986)), NASBA (Comptom, J., Nature, 650, 91-92 (1991)), TMA (Kacian, D. L., and Fultz, T. J., U.S. Pat. No. 5,399,491 (1995)), and SDA (Walker, G. T., Little, M. C., et al., Proc. Natl. Acad. Sci., U.S.A., 89, 392-396 (1992)). Gene fragments amplified by means of, for example, PCR may be isolated and purified through a conventional technique such as gel electrophoresis. Alternatively, purification of such gene fragments may be performed by use of a column. The gene fragment purification can be confirmed through, for example, mass spectrometry or electrophoresis. In accordance with properties of the thus-amplified gene fragments, the gene fragments are applied for detection of the A1A1 genotype of DRD2 TaqIA polymorphism employed in the present invention.

Detection of Polymorphism

According to the method of the invention, DNA located in genomic region of specific gene in aforementioned sample is sequenced and analyzed to detect the presence or absence of SNP (determination of SNP). Specifically, this detection can be performed through, for example, any of the below-described methods (1) through (8).

(1) Direct Nucleotide Sequencing

Detection of a polymorphism(s) can be performed by determining DNA sequences of a specific gene through a direct nucleotide sequencing method, which has conventionally been employed for sequencing of such a gene; for example, the dideoxy method (Sanger, et al., Proc. Natl. Acad. Sci., U.S.A., 74, 5463-5467 (1977)) or the Maxam-Gilbert method [Methods in Enzymology, 65, 499 (1980)]. The genetic polymorphism detection may be performed through a combination of such a direct nucleotide sequencing method and a DNA amplification method (e.g., PCR). Particularly, a combination of such a direct nucleotide sequencing method and PCR or a similar DNA amplification method is preferred, since this combination needs only a small amount of a DNA sample, and enables simple and easy detection with high sensitivity and accuracy.

Basically, this preferred method can be performed by, for example, performing direct nucleotide sequencing on a PCR-amplified genomic DNA fragment or a purified product thereof using the dideoxy method, the Maxam-Gilbert method, or a similar method. For the sake of convenience, the preferred method can be performed through nucleotide sequencing by use of, for example, a commercially available sequencing kit. Thus, the presence of polymorphisms at the aforementioned specific genomic DNA sites of a human gene can be detected.

In the aforementioned method and the below-described methods, no particular limitation is imposed on the PCR-amplified DNA fragment, so long as the DNA fragment includes at least one of the aforementioned specific sites at which polymorphisms is expected to occur. The DNA fragment generally has a length of about 50 to several thousands of bp, preferably 50 to several hundreds of bp.

(2) Allele-Specific Oligonucleotide Dot Blot Method

Alternatively, detection of a specific genetic polymorphism(s) can be performed through the allele-specific oligonucleotide (ASO)-dot blot method (Conner, B. J., et al., Proc. Natl. Acad. Sci., U.S.A., 80, 278-282 (1983)). This method can be performed through, for example, dot blot analysis in which a PCR-amplified gene fragment by use of a forward primer and reverse primer designed so as to sandwich a target is hybridized with an allele-specific oligonucleotide probe containing SNP site. Thus, the presence of SNP in the gene fragment can be determined.

(3) Single nucleotide primer extension assay

Detection of a specific genetic polymorphism(s) can also be performed through a single nucleotide extension assay, such as the SNaPshot assay, pyrosequencing, or the point mutation detection assay disclosed in Japanese Patent Application Laid-Open (kokai) No. 2000-279197. In such an assay, a probe designed so as to correspond to a nucleotide immediately (or several nucleotides) before a target polymorphism (SNP) (i.e., a probe designed such that the 3′-end thereof corresponds to one (or several) nucleotide upstream of the polymorphism) is annealed to a DNA sample. Each of the aforementioned assays can be performed by use of a commercially available SNPs detection kit and the software attached to the kit.

For example, the SNaPshot assay can be performed by use of ABI PRISM SNaPshot ddNTP Primer Extension Kit (PE Applied Biosystems). Detection of SNPs can be performed through detection and analysis of fluorescent fragments generated after reaction by use of ABI PRISM 310/377/3100/3700 DNA Analyzer (PE Applied Biosystems) and GeneScan software.

Pyrosequencing can be performed through, for example, the following procedure. Specifically, genomic DNA is isolated from, for example, a blood sample through a conventional method; several tens to several hundreds of nucleotides (including a polymorphism) are PCR-amplified by use of a biotin-labeled primer; single-stranded DNA is purified by use of magnet beads; and the thus-purified DNA is employed as a sample. A primer designed to have a complementary sequence corresponding to several nucleotides upstream of a target polymorphism is annealed to the sample, and then each dNTP is added to the mixture one after another according to the sequence in the vicinity of the polymorphism input in software. Pyrophosphoric acid (PPi) released from nucleotide extension of DNA polymerase is converted to ATP by ATP sulfurylase, and luciferase generates detectable light using this ATP, which can be detected with a chemiluminescence detector, a CCD camera, etc. Thus, genotyping can be performed through analysis of the peak of luminescence obtained through addition of the dNTPs. This method enables genotyping in about 15 minutes for 96 samples.

The aforementioned method can use a generally employed reagent and apparatus. Examples include reagents such as commercially available SNP Reagent Kits (Pyrosequencing AB) which contain, as components, a mixture of the following four enzymes: DNA polymerase, ATP-sulfurylase, luciferase, and apyrase, a substrate solution containing luciferin and APS (adenosine 5′-phosphosulfate), and dNTPs containing dATP (deoxyadenosine 5′-triphosphate), dCTP, dGTP, and dTTP; and apparatuses such as PSQ96 system for automatic DNA sequence analysis (Pyrosequencing AB); and SNP software employed for the analysis (Pyrosequencing AB).

Alternatively, pyrosequencing can be performed through, for example, the method described in U.S. Pat. No. 6,159,693. Specifically, an isolated genomic DNA is amplified by PCR; the thus-amplified PCR product is purified; and the resultant product is reacted with pyrophosphoric acid by use of READIT System (Promega Corporation), followed by analysis of the resultant data.

(4) PCR-Single Strand Conformation Polymorphism (SSCP) Analysis

The method of the present invention can employ the PCR-SSCP method (Orita, M., Iwahara, H., et al., Proc. Natl. Acad. Sci., U.S.A., 86, 2776-2770 (1989)). In this method, an amplified PCR product (single-stranded DNA) is subjected to non-denatured polyacrylamide gel electrophoresis, and the presence of single nucleotide polymorphims is determined on the basis of the mobility difference.

(5) PCR-Restriction Enzyme Fragment Length Polymorphism (RFLP) Analysis

In the present invention, in the case where, for example, a nucleotide sequence including a polymorphims, which are targeted for detection of SNPs or haplotype of a specific gene, contains a restriction enzyme recognition site, the detection can be performed through restriction enzyme fragment length polymorphism analysis (RFLP analysis: Botstein, D. R., et al., Am. J. Hum. Gen., 32, 314-331 (1980)).

An RFLP method can be specifically performed, for example, as follows, depending on the genotype at the specific position of the genetic polymorphism described in the TaqIA restriction enzyme polymorphism in the DRD2. For example, taking the genotype at the specific position of the TaqIA restriction enzyme polymorphism in the DRD2 as an example, in order to detect the genetic polymorphism in which the genotype at position 32806 in genomic sequence AF050737 including the DRD2 gene is T/T, the analysis is performed using restriction enzymes capable of recognizing the sequence including the genotype of interest and the before and after sequences thereof. The enzymes used for such an RFLP could be publicly known various restriction enzymes capable of recognizing the sequence including the objective genotype and the before and after sequences thereof. The RFLP analysis is more preferably done as PCR-RFLP analysis; i.e., analysis performed on a large amount of sample DNA which has been amplified and prepared in advance through PCR or a modification thereof. Thus, the presence of polymorphism can be detected on the basis of the presence of a specific cleavage site.

In detecting a polymorphism(s) according to this method, firstly, the genomic DNA is extracted from a human biological sample, and a DNA fragment of the region including a polymorphism of the gene is amplified, for example by PCR, thereby preparing a large amount of a DNA sample. Subsequently, the amplified DNA sample is digested by use of a specific restriction enzyme, and DNA cleavage patterns (e.g., the presence of cleavage, or the base length of cleaved fragments) are confirmed through a conventional method such as gel electrophoresis.

(6) Invader Assay

In the invention, detection of SNPs of a specific gene(s) can also be performed through the Invader assay. The Invader assay can be performed with reference to the following publications: Lyamichev, V., et al., Nat. Bioltechnol., 17(3) 292-296 (1999); and International Patent Publication WO 9823774 (Japanese Kohyo Patent Publication No. 2001-526526). The Invader assay enables analysis of SNPs of genomic DNA without necessity of pre-amplification of target DNA. For example, the Invader assay is performed as follows.

In order to detect whether SNPs of the objective specific gene, e.g., the TaqIA polymorphism in the DRD2 are present or not, first genomic DNA is isolated. Subsequently, a first target probe composed of a 5′-flap composed of 15 to 50 base length and an oligonucleotide composed of 30 to several hundred bases synthesized so that a nucleotide to be detected (SNP in the present invention) is disposed at 3′ end of the 5′-flap and nucleotides other than the nucleotide of the objective genotype are complementary to the target genomic DNA, and an invader oligonucleotide probe composed of 15 to several ten base length so that the nucleotide complementary to the nucleotide to be detected is disposed at 3′ end and the nucleotides other than it are complementary to the target genomic DNA are synthesized by, for example, an automatic synthesizer. The isolated genomic DNA and the enzyme (flap endonuclease) to cleave the 5′-flap of the first probe are simultaneously added to these probes, and reacted in an appropriate reaction solution.

If the genomic DNA in the specimen has the desired genetic polymorphism (SNP), a first reaction to liberate the 5′-flap having the nucleotide of the desired genotype at 3′ end is terminated. If the genomic DNA in the specimen does not have the nucleotide sequence of the desired genotype, the cleavage by the above enzyme does not occur. The 5′-flap cleaved by the enzyme and liberated from the first probe is complementarily bound as the target to fluorescence resonance energy transfer (FRET) probe, and the 3′ end of the 5′-flap invades in the FRET probe (invasion). Likewise, the reaction by the enzyme occurs and quenched fluorescent dyestuff is liberated.

Then, each FRET probe used for a second reaction contains the identical sequence regardless of being the target to be detected, and is constructed to be essentially composed of the following two elements.

(1) a 3′ region which is complementary to a product cleaved through the first reaction; and (2) a self-complementary region which forms a duplex for mimicking a single-stranded probe, which is hybridized with a target, and which contains a reporter fluorescent dye and a quencher fluorescent dye.

When the reporter fluorescent dye and the quencher fluorescent dye are bound to the same probe, the fluorescence intensity of the reporter fluorescent dye is quenched through fluorescence resonance energy transfer. Whereas when the reporter fluorescent dye and the quencher fluorescent dye are not bound to the same probe, the fluorescence intensity of the reporter fluorescent dye is not quenched. When the 5′ flap released from the cleaved first probe is hybridized with the FRET probe, the resultant product acts as an invader oligonucleotide in the second reaction, and an invasion complex that is recognized by the enzyme is produced. Thus, cleavage of the FRET probe by the aforementioned enzyme separates the two fluorescent dyes, thereby yielding a detectable fluorescent signal. The signal can be read by use of, for example, a standard fluorescence microtiter plate reader, whereby the presence of target SNPs (genetic polymorphism) can be detected. A combination of the first and second reactions can amplify the signal by a factor of 1×10⁶. Employment of two FRET probes having different fluorescent dyes also enables detection or typing of SNP.

(7) Quantitative Real-Time PCR Assay

In the invention, detection of polymorphisms of a specific gene(s) can also be readily performed by quantitative real-time PCR assay (TaqMan assay). This assay can be performed through, for example, the following procedure. Specifically, firstly, to confirm genetic polymorphism of the target SNPs, a DNA fragment is prepared as a forward primer or reverse primer formed of, for example, 15 to 39 nucleotides to detect DNA fragments in appropriate region containing the polymorphism (nucleotide site). In this case, the forward primer or reverse primer is prepared so as not to contain the polymorphims. Subsequently, there is prepared a probe which has both a reporter fluorescent dye and a quencher fluorescent dye, and the probe contains, for example, a 15 to 50 by oligonucleotide which correspond to a partial sequence of amplified fragment. The nucleotide sequence of the probe has to be selected such that a region with which both of the forward and reverse primer do not hybridize. The probe is designed so as to have a sequence complementary to an allele-specific sequence for detecting the presence of a target single nucleotide polymorphism. By use of the probe, a target DNA fragment of a specific gene of a sample to be detected, for example, DRD2 gene containing the TaqIA polymorphism is amplified through PCR, and fluorescence from the resultant reaction mixture is real-time measured. Thus, the presence of polymorphism can be detected. Employment of two probes having different fluorescent dyes also enables detection of both alleles.

The reporter fluorescent dye employed in the aforementioned Invader assay or TaqMan assay is preferably a fluorescein fluorescent dye such as FAM (6-carboxy-fluorescein), whereas the quencher fluorescent dye is preferably a rhodamine fluorescent dye such as TAMRA (6-carboxy-tetramethyl-rhodamine). These fluorescent dyes are known, and are contained in commercially available real-time PCR detection kits. In the present invention, such a commercially available fluorescent dye can be employed. No particular limitation is imposed on the binding position of the reporter fluorescent dye or the quencher fluorescent dye, but generally, the reporter fluorescent dye is bound to one end (preferably the 5′-end) of the oligonucleotide constituting the probe, and the quencher fluorescent dye is bound to the other end. The method for binding a fluorescent dye to an oligonucleotide is known, and is described in, for example, Noble, et al., (1984), Nuc. Acids Res., 12: 3387-3403 or Iyer, et al., (1990), J. Am. Chem. Soc., 112: 1253-1254.

The TaqMan assay per se is known, and apparatuses and kits for the TaqMan assay are commercially available. In the present invention, such a commercially available apparatus or kit can be employed. These apparatuses and kits are employed in carrying out the invention, for example, according to the method described in Japanese Patent No. 2,825,976, or according to the ABI PRISM 7700 sequencing system user manual (PE Applied Biosystems).

(8) Genetic Polymorphism Assay Employing a Mass Spectrometer (Mass Array)

The mass array assay detects the difference in molecular weight between polymorphisms. Specifically, a region including a polymorphism to be detected is amplified through PCR, and then an extension primer is hybridized with a sequence immediately before the position of SNP, followed by extension reaction by use of a reaction mixture containing a ddNTP/dNTP mixture (e.g., a reaction mixture containing ddATP, dCTP, dGTP, and dTTP), thereby yielding a fragment having a length depending on the type of SNP. The resultant fragment is purified, and then subjected to analysis by use of, for example, a MALDI-TOF mass spectrometer, whereby the relationship between the molecular weight and the genetic polymorphism can be analyzed (Pusch, W., Wurmbach, JH., Thiele, H., Kostrzewa, M., MALDI-TOF mass spectrometry-based SNP genotyping, Pharmacogenomics, 3 (4): 537-48 (2002)). This assay can be readily performed by use of, for example, Sequenom Mass ARRAY high throughput SNP analysis system (http://www.sequenom.com/Files/applications/hme_assay.html).

(9) Other Detection Methods

Detection of SNPs of a gene(s) used in the invention can also be performed through, for example, any of the methods which have conventionally been known as DNA sequencing methods or polymorphisms or mutation detection methods. Examples of the methods are listed as below.

(9-1) PCR-SSO method employing sequence-specific oligonucleotide

A method in which a probe for SNPs is immobilized on a carrier; a sample (gene amplified product) is hybridized with the probe; and a difference in hybridization efficiency is determined on the basis of the presence of mismatch.

(9-2) PCR-SSP method for point mutation detection

A method by use of a sequence-specific primer for gene amplification which is designed such that a nucleotide corresponding to point mutation becomes the 3′-end nucleotide, which method utilizes that a significant difference in PCR amplification efficiency occurs depending on the complementarity of the 3′-end nucleotide of the primer.

(9-3) PCR-DGGE (denaturing gradient gel electrophoresis)

When DNA fragment including a polymorphism is hybridized with a wildtype DNA fragment, and then the thus-hybridized product is electrophoresed on a polyacrylamide gel with gradually increasing denaturant (e.g., urea or formamide) concentrations, the product is converted into single-stranded DNA fragments at a position of lower denaturant concentration, as compared with the case of non-mismatched homogenous double-stranded DNA fragments. The single-stranded DNA fragments migrate at a rate higher than the migration rate of the double-stranded DNA fragments, and therefore single nucleotide polymorphism can be detected through comparison of the mobilities of the DNA fragments.

(9-4) PCR-DGGE/GC Clamp Method (Shefield, V. C., et al., Proc. Natl. Acad. Sci., U.S.A., 86, 232-236 (1989))

This method is a modification of the aforementioned PCR-DGGE, in which a region having a high GC content is added to a target DNA fragment for detection of a polymorphism(s). This method compensates for the disadvantage of the PCR-DGGE in detection of substitution, deletion, addition, or insertion of polymorphism nucleotides. This method requires a step of adding a GC clamp to a target DNA fragment for polymorphism detection.

(9-5) RNase protection assay (Finkelstein, J., et al., Genomics, 7, 167-172 (1990))

(9-6) In situ RT-PCR (Nucl. Acids Res., 21, 3159-3166 (1993))

(9-7) In situ hybridization

(9-8) Southern blotting (Sambrook, J., et al., Molecular Cloning a Laboratory Manual., Cold Spring Harbor Laboratory Press: NY. (1989))

(9-9) Dot hybridization assay (see, for example, Southern, E. M., J. Mol. Biol., 98: 503-517 (1975))

(9-10) Fluorescence in situ hybridization (FISH: Takahashi, E., et al., Hum. Genet., 86, 1416 (1990))

(9-11) Comparative genomic hybridization (CGH: Kallioneimi, A., et al., Science, 258, 818-821 (1992)), (Spectral karyotyping: SKY: Rowley, J. D., et al., Blood, 93, 2038-2042 (1999))

(9-12) Method employing yeast artificial chromosome (YAC) vector clone as a probe (Lengauer, C., et al., Cancer Res., 52, 2590-2596 (1992)).

Thus, polymorphisms (SNPs) or haplotype of the human gene used in the invention can be detected.

It is predicted that the possibility is high that high improvement effects of the aripiprazole treatment on patients with various psychoneurotic symptoms of the psychiatric disorder bring out when those patients with the aripiprazole treatment have the genetic polymorphism identified as the marker in this invention.

In those subjected to the aripiprazole treatment, who were determined to have high possibility that the improvement effect on various psychoneurotic symptoms in the psychiatric disorder appears when aripiprazole is dosed in this way, considering the possibility that the improvement effect on various psychoneurotic symptoms in the psychiatric disorder appears when aripiprazole is dosed, it is possible to discuss whether the aripiprazole treatment is started from a low dose or whether another psychotropic drug is changed to aripiprazole, and thus, a chance of administering an unnecessary drug for the patient is reduced, consequently leading to the reduction of the appearance of side effects and accidents due to the drug administration.

In particular, the genetic polymorphism of the human gene detected according to the method of the present invention is highly associated with the possibility that the improvement effects on psychiatric disorder including various psychoneurotic symptoms when aripiprazole is treated. Therefore, based on its detection result, the personalized therapy for an individual subjects to the aripiprazole treatment becomes possible, i.e., it becomes possible to perform the therapy in which the drug which has the highest efficacy for the individual patient is properly selected.

Oligonucleotide

The present invention also provides an oligonucleotide serving as a primer or probe for detection of the genetic polymorphism, which is used in the determination (detection) method of the present invention. No particular limitation is imposed on the oligonucleotide, so long as it can specifically amplify a region including specific polymorphisms of the human gene. The oligonucleotide can be constructed on the basis of sequence data of each specific polymorphism and synthesized by conventional methods.

More specifically, the oligonucleotide can be synthesized by a generally employed chemical synthesis method such as the phosphoroamidite method or the phosphotriester method, or can be synthesized by use of a commercially available automated oligonucleotide synthesis apparatus such as Pharmacia LKB Gene Assembler Plus (product of Pharmacia). A double-stranded fragment can be obtained by annealing of a chemically synthesized single-stranded oligonucleotide and its complementary strand under appropriate conditions, or synthesized by using an appropriate primer and DNA polymerase.

Preferred examples of the aforementioned oligonucleotide serving as a probe or primer include partial oligonucleotides corresponding to a DNA fragment designed so as to contain a polymorphism of a specific gene. These oligonucleotides have at least a sequence of 10 bases (generally about 10 to 35 a sequence of bases). The oligonucleotide serving as a primer pair may be oligonucleotides having two sequences which are designed and synthesized so as to detect SNP in the DNA sequence of the gene. A DNA fragment containing a polymorphism per se may be used as the oligonucleotide serving as a probe.

Preferred examples of the aforementioned oligonucleotide serving as a probe or primer include oligonucleotides shown below. Preferred oligonucleotides contain the SNP site and one or more bases upstream thereof and have at least a sequence of 15 bases. The genetic polymorphism is detected by using a probe or primer wherein said probe or primer have an oligonucleotide having the sequence of SEQ NO:3, or at least 10 nucleotides wherein the polymorphism located at position 32806 in genomic sequence AF050737 including the DRD2 gene is positioned at the 3′ terminal or 1 to a few bases downstream from the 3′ terminal in the oligonucleotides.

Specific examples of oligonucleotides of the invention include a forward primer (SEQ ID No. 4) and a reverse primer (SEQ ID No. 5) for aforementioned genes as shown in examples. Gene specific probes of the invention include a probe (SEQ ID No. 3) for detecting A1A1 of TaqIA polymorphism in the Dopamine D2 Receptor.

Kit for Determination

The determination (detection) method of the present invention can be more easily performed by use of a reagent kit for detecting SNPs and determining the genotypes of a specific human gene of a sample. The present invention also provides a kit for such determination. One example of kit of the invention includes, as an essential component, at least a DNA fragment which hybridizes with a partial or entire nucleotide sequences or its complementary sequences including any of aforementioned SNPs of the human gene, or which hybridizes with a sequence containing an oligonucleotide with one base or several bases upstream of the polymorphic site.

Other components of the kit of the present invention are, for example, a labeling reagent, and reagents required for PCR (e.g., Taq DNA polymerase, deoxynucleotide triphosphate, or a primer for DNA amplification). Examples of the labeling reagent include chemical modification substances such as a radioactive isotope, a light-emitting substance, and a fluorescent substance. The DNA fragment per se may be conjugated in advance with such a labeling reagent. The kit of the present invention may further include, for example, appropriate reaction diluents, standard antibodies, buffers, detergents, or reaction stopping solutions, to perform measurement conveniently.

The genetic polymorphism in the specific gene found by the present inventor is highly associated with the possibility that the improvement effects of the aripiprazole treatment on the various psychoneurotic symptoms of the psychiatric disorder bring out. Thus, according to the present invention, for improving the various psychoneurotic symptoms having the psychiatric disorder, in the personalized medicine for the individual subject to the aripiprazole use, it becomes possible to properly select the drug which is highly safe for the patient.

The present invention provides a method for detecting such a human specific genetic polymorphism as the marker associated with the high improvement effects of the aripiprazole treatment on the various psychoneurotic symptoms of the psychiatric disorder, i.e., the method for detecting the specific genetic polymorphism in the specimen obtained from those subjects with the aripiprazole treatment as the marker for identifying the improvement effects on the various psychoneurotic symptoms of the psychiatric disorder, as well as a diagnostic reagent and a diagnostic kit used for the method.

The present invention provides the method for detecting the marker for identifying the high improvement effects of the aripiprazole treatment on the various psychoneurotic symptoms of the psychiatric disorder, particularly the method for determining the presence of the high improvement effects of the aripiprazole treatment on the various psychoneurotic symptoms of the psychiatric disorder by detecting the specific genetic polymorphism in the specimen from those subjects with the aripiprazole treatment, a kit therefor, the genetic polymorphism and the genotype utilized for them, primers and probes for detecting the genotype. These are useful for an order choice for selecting the drug to be administered in personalized medicine for the individual patient.

### EXAMPLE

The invention is described below in detail using examples. However, the invention is in no way limited by the examples.

### Example 1

### 1) Subjects

A total of 90 patients with schizophrenia, schizophreniform disorder or schizoaffective disorder were enrolled in the present study after each patient had signed an informed consent.

Men and nonpregnant, nonlactating women aged 18 to 65 years who met DSM-IV criteria for schizophrenia, schizophreniform disorder or schizoaffective disorder with acute episode (duration of the present episode ≦4 weeks) were eligible for enrollment in the present study.

For inclusion, patients had to consent participation in pharmacogenetic study of APLUS and have a Positive and Negative Syndrome Scale (PANSS) total score of at least 60, and a minimum score of 4 (moderate) on at least 2 items of 4 PANSS items (hallucinatory behavior, delusions, conceptual disorganization, and suspiciousness).

Exclusion criteria included a psychiatric disorder other than schizophrenia, schizophreniform disorder or schizoaffective disorder requiring pharmacotherapy; patients with violent behavior; a recent history of suicide attempt or serious suicide ideation; neurological abnormality other than tardive dyskinesia or extrapyramidal symptoms induced by antipsychotic drugs; current diagnosis of psychoactive substance dependence or a history of substance or alcohol abuse (DSM-IV) within 1 month of the start of the study; patients who require any medication of carbamazepine, valproic acid, lithium or any drug known to inhibit the activity of cytochrome P450 2D6 or 3A4 enzyme; administration of long-acting antipsychotic drugs before the study registration; medical condition disturbing absorbance of the study drug; patient with somatic symptoms which may be misperceived as a psychotic symptoms or adverse effects induced by antipsychotic drugs; a clinically significant laboratory abnormality; administration of an investigational drug within 4 weeks before the start of the study; a history of participation in any clinical trial on aripiprazole; or any other acute or unstable medical condition.

### 2) Target Genotype

Taq IA polymorphism on human dopamine D2 receptor (DRD2) was selected, wherein said Taq IA polymorphism includes the homozygous wild type (A2/A2), heterozygous (A2/A1) and homozygous variant-type (A/A) groups for this study.

### 3) DRD2 Taq1A Genotyping

Genomic DNA was extracted from the peripheral whole blood of each of the subjects with a Qiagen DNA extraction kit (Qiagen, Hilden, Germany) in accordance with the manufacture's instructions. The presence of the A1 and A2 allele was determined via PCR and single base extension, using SnaPshot analysis. The following primers were used for PCR amplification:

Forward primer shown in SEQ ID NO: 4
(5′-gctggccaagttgtctaaat-3′)
Reverse primer shown in SEQ ID NO: 5
(5′-tggagctgtgaactggact-3′)

For SnaPshot analysis, the PCR products were purified using exonuclease I and shrimp alkaline phosphatase (USB, Cleveland, Ohio, USA) and then mixed with AmpliTaq DNA polymerase, four fluorescent-labeled dideoxynucleotides, each of the primers for single base extention, and the reaction buffer of an ABI PRISM SnaPshot™ Multiplex Kit (Applied Biosystems), in accordance with the manufacture's protocols. The primer used for single base extension was shown in SEQ ID NO: 3 (5′-cacagccatcctcaaagtgctggtc-3′) for Taq IA polymorphism.

This primer was extended over 25 cycles of 96° C. for 10 sec, 50° C. for 5 sec, and 60° C. for 30 sec. The amplicons were then analyzed using an ABI Prism 3700 Autimated Sequencer (Applied Biosystems). DNA sequences proximal to the polymorphic sites were verified via direct sequencing.

### 4) Assessment

Treatment efficacy was assessed using the Positive and Negative Syndrome Scale (PANSS). Efficacy evaluations were performed at screening and at weeks 1, 2, 3, 4, 6, 8, 12, 16 and 26.

### 5) Statistical Analysis

The primary efficacy parameter was the reduction ratio from baseline in PANSS total score. To determine the effects of the DRD2 TaqIA polymorphism on an efficacy of aripiprazole in the treatment of schizophrenia and schizophrenia-related disorders, we compared data from the patients homogenous for minor allele (A1A1) with those from the patients with other genotypes (A1A2, A2A2).

To compare efficacy after treatment stabilization, data at screening and week 8, 12, 16, 26 were analyzed.

For repeated measures analysis, mixed effects model was employed. The correlation of the repeated measures was modeled via a random subject intercept and a first-order autoregressive covariance matrix.

### IV. The Relationship Between Gene and Clinical Response

These days, the study on the relationship between gene and clinical response gets a lot of attention.

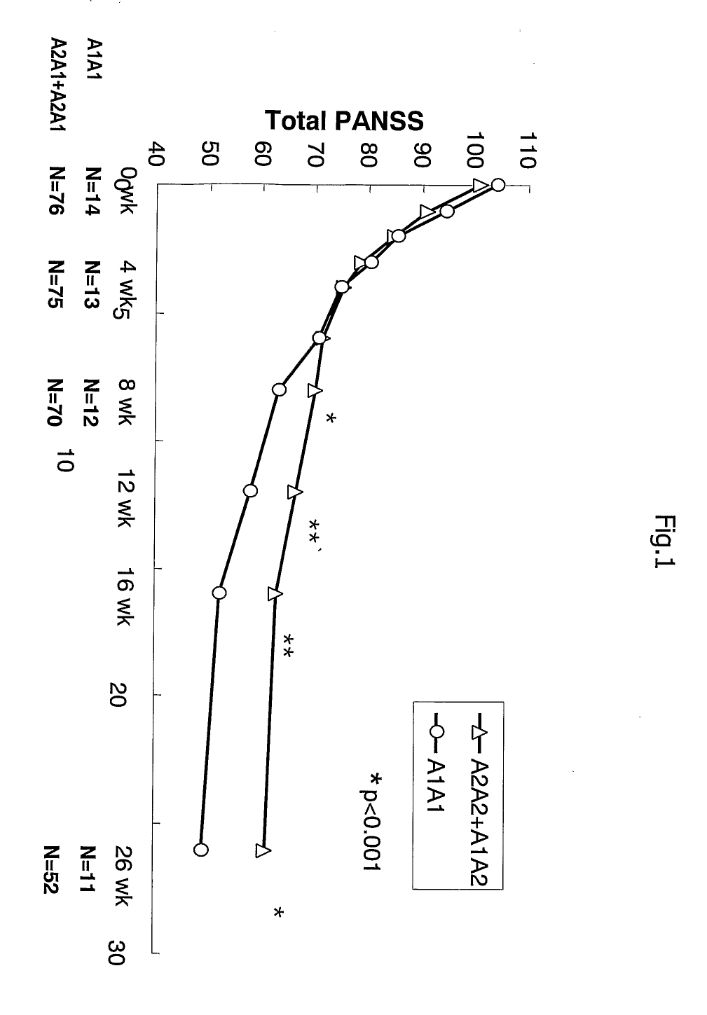

Given this factor, the genotyping of patients was performed by using dopamine D₂ receptor Taq IA alleles and thereby patients were classified into homozygous wild type A2A2, heterozygous type A1A2 or homozygous mutant type A1A1. An interesting result was obtained when responses against aripiprazole were analyzed by PANSS total score. The results are shown in FIG. 1.

The results indicated that symptoms of patients having A1A1 alleles dramatically improved at 8, 12 and 16 weeks compared to those of patients having A1A2 and A2A2 alleles. That means that the reaction against aripiprazole varies by the type of dopamine D₂ receptor Taq IA alleles.

These results are very surprising since it is unusual that the difference of reaction among various genotypes would be recognized in clinical tests.

The significant improvement of PANSS score (0 week vs 8, 12, 16 and 26 weeks) was observed at 8 weeks or later in the group having minor alleles of Taq IA polymorphism (A1A1 types) compared to the group having major alleles (A1A2 types and A2A2 types). The difference between two groups was observed also at the end of duration period after lapse of 26 weeks. The results are shown in FIG. 1. In addition, table 1 shows demographic data.

TABLE 1
Demographic data

| genotype | A1A1 | A1A2 + A2A2 | p value |
|---|---|---|---|
| subject (n) | 14 | 76 | |
| sex (%) | | | p > 0.05 |
| male | 42.9 | 40.8 | |
| female | 57.1 | 59.2 | |
| age ± SD (yr) | 38.6 ± 11.0 | 37.8 ± 9.7 | p > 0.05 |
| Diagnosis (%) | | | p > 0.05 |
| schizophreniform | 7.1 | 3.9 | |
| schizophrenia | 85.8 | 96.1 | |
| schizoaffective | 7.1 | 0.0 | |
| Episode (%) | | | p > 0.05 |
| first | 50.0 | 34.2 | |
| recurrent | 50.0 | 65.8 | |
| Previous medication (%) | | | p > 0.05 |
| typical AP | 57.1 | 34.0 | |
| clozapine | 0.0 | 2.0 | |
| risperidone | 28.6 | 30.0 | |
| olanzapine | 0.0 | 14.0 | |
| quetiapine | 0.0 | 12.0 | |
| amisulpiride | 14.3 | 2.0 | |
| others | 0.0 | 6.0 | |
| Illness duration ± SD | 116.9 ± 131.5 | 97.0 ± 94.4 | p > 0.05 |
| (month) | | | |
| Number of admission | 3.3 ± 3.2 | 2.9 ± 2.7 | p > 0.05 |

### Benefit/Application

Inter-individual variation in drug response to antipsychotic drugs has remained critical in the treatment of patients with schizophrenia. The present invention can predict clinical response to aripiprazole in patients with schizophrenia and provide criteria by which to select antipsychotic drugs, and improve therapeutic efficiency in the treatment of schizophrenia before administration of antipsychotic medication.

### Sequence Listing Free Text

SEQ ID No. 3-5: probe or primer sequences

1. A method of identifying a marker responsive to aripiprazole treatment comprising the steps of:
(a) preparing a genomic DNA Dopamine D2 Receptor (DRD2) gene or its complementary strand from a specimen of a subject with a psychiatric disorder;
(b) analyzing a sequence of said genomic DNA or its complementary strand to determine the genotype of the TaqIA polymorphism in the DRD2 gene; and
(c) determining the proposed high therapeutic effects of aripiprazole treatment on a subject with psychiatric disorder by using the polymorphism as a marker.

2. The method according to claim 1, wherein the genotype of TaqIA in the DRD2 gene is at least one genotype selected from the group consisting of A1A1, A1A2 and A2A2.

3. The method according to claim 1, wherein said genetic polymorphism of TaqIA in the DRD2 gene (DRD2 TaqIA polymorphism) is reference dbSNP ID NO: rs1800497.

4. The method according to claim 1, wherein said psychiatric disorder is selected from Schizophrenia, Schizophreniform disorder, or Schizoaffective disorder.

5. The method according to claim 1, wherein said marker is at least one genetic polymorphism selected from the group consisting of (a) to (c);
(a) a polymorphism that is T/T (A1A1 type);
(b) a polymorphism that is T/C (A1A2 type); and
(c) a polymorphism that is C/C (A2A2 type);
wherein said polymorphism is located at position 32806 in AF050737 which includes DRD2 gene.

6. The method according to claim 1, wherein the genetic polymorphism is detected through at least one technique selected from the group consisting of direct nucleotide sequencing, allele-specific oligonucleotide (ASO)-dot blot analysis, single nucleotide primer extension assay, PCR-single strand conformation polymorphism (SSCP) analysis, PCR-restriction enzyme fragment length polymorphism (RFLP) analysis, Invader assay, quantitative real-time PCR assay, and genetic polymorphism assay employing a mass spectrometer (mass array).

7. The method according to claim 6, wherein the genetic polymorphism is detected by using single nucleotide primer extension assay.

8. The method according to claim 6, wherein the genetic polymorphism is detected by using a probe or primer which has an oligonucleotide having the sequence of SEQ ID NO:3, or at least 10 nucleotides thereof wherein said oligonucleotide sequences defining the 3′terminal position at 1 to a few base from the position of the polymorphism located at position 32806 in AF050737.

9. The method according to claim 6, wherein the PCR-restriction enzyme fragment length polymorphism (RFLP) analysis is performed by using the restriction enzyme (Taq I) for detecting T/T, T/C, or C/C located at position 32806 in AF050737.

10. A method of determining a therapeutic effect of aripiprazole on patients with psychoneurotic diseases comprising the steps of:
(a) preparing a genomic DNA Dopamine D2 Receptor (DRD2) gene or its complementary strand from a specimen of a patient with a psychiatric disorder;
(b) analyzing a sequence of said genomic DNA or its complementary-strand to determine the genotype of the TaqIA polymorphism in the DRD2 gene; and
(c) determining the proposed high therapeutic effects of aripiprazole treatment on a subject with psychiatric disorder by using the polymorphism as a marker.
